(19) United States
(12) Patent Application Publication
(10) Pub. No.: US 2008/0132966 A1
(43) Pub. Date: Jun. 5, 2008

(54) STIMULATION OF CORONARY ARTERY BARORECEPTORS

(75) Inventors: Howard Levin, Teaneck, NJ (US); Mark Gelfand, New York, NY (US)

Correspondence Address: NIXON & VANDERHYE, PC, 901 NORTH GLEBE ROAD, 11TH FLOOR, ARLINGTON, VA, 22203, US

(73) Assignee: G&L CONSULTING, LLC, New York, NY (US)

(21) Appl. No.: 11/950,911

(22) Filed: Dec. 5, 2007

Related U.S. Application Data
(60) Provisional application No. 60/868,575, filed on Dec. 5, 2006.

(51) Int. Cl. A61N 1/00

(52) U.S. Cl. 607/17; 607/14; 607/119

(57) ABSTRACT

A method and apparatus for treatment of hypertension by electric stimulation of coronary artery baroreceptors wherein stimulation electrodes are implanted in the left coronary artery, periarterialy or in an adjacent coronary vein. Stimulation is non excitatory and does not cause heart contractions. Stimulation can be applied during the ventricular refractory period resulting in baroreceptor activation and subsequent reduction of blood pressure. An implantable device monitors ECG and applies to a field of stimulation of baroreceptors.

### RELATED APPLICATION

This application claims the benefit of the filing date of U.S. Provisional Patent Application Ser. No. 60/868,575, filed Dec. 5, 2006, the entirety of which application is incorporated by reference.

### BACKGROUND

The present invention generally relates to implantable devices for nerve stimulation and pacing therapy, and more particularly, the present invention is concerned with cardiac therapies involving the controlled delivery of electrical stimulations to the heart baroreceptors in order to invoke natural baroreflex for the treatment of hypertension, and an apparatus for delivering such therapies with the objective of controlling blood pressure.

Hypertension

It is generally accepted that high blood pressure (HBP, also called hypertension) is bad, but most people don't know why, and what the term really means. In fact, all humans have high blood pressure some of the time, and we wouldn't be able to function if we didn't (such as during exercise). High blood pressure is only of concern when it persists for long periods of time or is extremely high over a very short (hours) period of time. Its adverse effects usually take many years to develop. Clinically important HBP is very common. According to official government figures, it affects 50 million people in the United States.

While everyone has high blood pressure some of the time, many people live their entire lives with moderately high blood pressure and never know it unless noticed on a routine visit to the doctor. Unfortunately, not all people are so lucky to detect high blood pressure early and obtain treatment. When undetected and not treated, high blood pressure significantly increases the risk of a number of serious events, mainly strokes and heart attacks.

The damage caused by high blood pressure is of three general sorts. The first is the one everyone thinks of—bursting a blood vessel. While this is dramatic and disastrous when it happens, it's actually the least common of the three problems. It occurs most frequently in the blood vessels of the brain, where the smaller arteries may develop a weak spot, called an aneurysm. This is an area where the wall is thinner than normal and a bulge develops. When there is a sudden surge of pressure the aneurysm may burst, resulting in bleeding into the tissues. If this occurs in the brain, it is called a stroke. In contrast, if this happens to the aorta (the main blood vessel in the body), it is called a ruptured aortic aneurysm. Both of these events can lead to permanent damage and death.

The second adverse consequence of high blood pressure is that it accelerates the deposition of cholesterol in the arteries forming a blockage. This problem, too, takes many years to develop, and it is very difficult to detect until it causes a major blockage. The most important sites to be affected are the heart, where the blockage can cause angina and heart attacks; the brain, where it causes strokes; the kidneys, where it causes renal failure (and can also make the blood pressure go even higher); and the legs, where it causes a condition known as intermittent claudication, which means pain during walking and may even lead to losing a limb.

Third, high blood pressure puts a strain on the heart. The heart has to work harder than normal to pump blood against a higher pressure. Due to the extra work, the heart muscle enlarges, just as any other muscle does when it is used excessively. Over a long period of time, the high blood pressure can lead to congestive heart failure, the most frequent cause for hospitalization in the United States. Whatever the underlying cause, when the blood pressure reaches a certain level for a sufficient length of time it sets off a vicious cycle of damage to the heart, brain, and kidneys, resulting in further elevation of the pressure.

Classification of hypertension by its severity is somewhat arbitrary because there is no precise level of pressure above which it suddenly becomes dangerous. Historically, blood pressure has been primarily classified according to the height of the diastolic pressure. Someone whose diastolic pressure runs between 90 and 95 mm Hg may be regarded as having borderline hypertension, a pressure between 95 and 110 mm Hg, it is considered moderate, and pressures at higher levels is termed severe. Recent data suggests that the systolic pressure is as, and maybe more important than, diastolic blood pressure in determining the patient's risk for serious adverse events. Systolic hypertension is mainly seen in people over the age of 65 and is characterized by a high systolic, but normal diastolic, pressure (a reading of 170/80 mm Hg would be typical). It's caused by an age-related loss of elasticity of the major arteries. Another form of HBP, Labile hypertension, is a commonly used term for describing people whose pressure is unusually labile or variable. The most dangerous type of HBP is called malignant hypertension or high blood pressure with evidence on physical exam that this pressure causing an acute deleterious affecting on vital organ function. Malignant hypertension is regarded as an emergency requiring immediate treatment in a hospital. Not surprisingly, if untreated, malignant hypertension can be rapidly fatal. Although more people are treated with drugs nowadays than before, malignant hypertension is still common.

The objective of treatment is not simply to lower the blood pressure, but to prevent its consequences, such as strokes and heart attacks. According to the American Heart Association high blood pressure is present in 50,000,000 Americans (Defined as systolic pressure 140 mm Hg or greater, and/or diastolic pressure 90 mm Hg or greater, or taking antihypertensive medication). Of those with HBP, 31.6 percent are unaware they have it; 27.4 percent are on medication and have it controlled; 26.2 percent are on medication but don't have their HBP under control; and 14.8 percent aren't on medication. In most cases, high blood pressure can be controlled with one or a combination of oral drugs. Of those patients that take medication to control HBP, many suffer from debilitating side effects of these drugs such as heart arrhythmias, inability to exercise or do normal activities of daily living and impotence.

Baroreflex

In cardiovascular physiology, the baroreflex or baroreceptor reflex is one of the body's homeostatic mechanisms for maintaining blood pressure. It provides a negative feedback loop in which an elevated blood pressure reflexively causes blood pressure to decrease; similarly, decreased blood pressure depresses the baroreflex, causing blood pressure to rise. The system relies on specialized neurons or biological stretch sensors (baroreceptors) in the aortic arch, carotid sinuses, and elsewhere to monitor changes in blood pressure and relay them to the brainstem. Subsequent changes in blood pressure are mediated by the autonomic nervous system.

The baroreceptors are stretch-sensitive mechanoreceptors. When blood pressure rises, the carotid and aortic sinuses are distended, resulting in stretch and therefore activation of the baroreceptors. Active baroreceptors fire action potentials (“spikes”) more frequently than inactive baroreceptors. The greater the stretch, the more rapidly baroreceptors fire action potentials. Baroreceptor signals are conducted to the brain by dedicated nerves. The end result of baroreceptor activation is inhibition of the sympathetic nervous system and activation of the parasympathetic nervous system.

The sympathetic and parasympathetic branches of the autonomic nervous system have opposing effects on blood pressure. Sympathetic activation leads to increased contractility of the heart, increased heart rate, venoconstriction, and arterial vasoconstriction, which tend to increase blood pressure by elevating both total peripheral resistance and cardiac output. Conversely, parasympathetic activation leads to a decrease in heart rate and a minor decrease in contractility, resulting in a decreased cardiac output and therefore a tendency to decrease blood pressure.

By coupling sympathetic inhibition with parasympathetic activation, the baroreflex maximizes its ability to reduce blood pressure. Sympathetic inhibition leads to a drop in total peripheral resistance and cardiac output, while parasympathetic activation leads to a depressed heart rate and reduced cardiac contractility. The combined effects will dramatically decrease blood pressure.

Similarly, coupling sympathetic activation with parasympathetic inhibition allows the baroreflex to elevate blood pressure effectively. Sympathetic activation increases total peripheral resistance and elevates cardiac output, the latter being enhanced by inhibition of the parasympathetic nervous system.

Most baroreceptors are tonically active at mean arterial pressures (MAP) above approximately 70 mm Hg, called the baroreceptor set point. When MAP falls below the set point, baroreceptors are essentially silent. The baroreceptor set point is not fixed; its value may change with changes in blood pressure that persist for 1-2 days. For example, in chronic hypertension, the set point may increase; on the other hand, chronic hypotension may result in a depression of the baroreceptor set point.

Carotid Sinus Baroreceptors Stimulation

Carotid sinus nerve stimulation is well known through developments at Medtronic, Inc., of Minneapolis, Minn. In the 1960s to early 1970s, Medtronic produced and marketed two carotid sinus nerve stimulators for treatment of hypertension, the “Barostat,” and angina, the “Angistat.” These devices lowered blood pressure, decreased myocardial work and oxygen consumption, and thereby alleviated hypertension and angina.

CVRx Inc. (Maple Grove, Minn.) is developing an implantable device to treat patients with high blood pressure based on stimulation of carotid sinus baroreceptors. This new device is called the RheoS™ Baroreflex Hypertension Therapy System. This system is made up of 3 major components: Rheos Implantable Pulse Generator, Rheos Carotid Sinus Leads, and Programmer.

The implantable pulse generator provides control and delivery of the activation energy through the carotid sinus leads. The leads conduct activation energy from the implantable pulse generator to the left and right carotid arteries. The Rheos programmer system provides the ability to non-invasively communicate with the Rheos pulse generator. A surgical implant procedure is used to place the pulse generator under the skin near the collarbone. The electrodes are placed on the carotid arteries and the leads run under the skin and are connected to the pulse generator.

The Rheos System works by electrically activating the baroreceptors, the sensors that regulate blood pressure. These baroreceptors are located on the carotid artery and in the carotid sinus. When activated by the Rheos System, signals are sent to the central nervous system and interpreted as a rise in blood pressure. The brain works to counteract this perceived rise in blood pressure by sending signals to other parts of the body to reduce blood pressure, including the heart, kidneys and blood vessels. According to CVRx, the Rheos system provides a physiological rational approach to reducing high blood pressure by allowing the brain to direct the body's own control mechanisms.

Coronary Artery Baroreceptors

Evidence suggesting the presence of coronary artery baroreceptors (CAB) on coronary arteries has existed for over 30 years. Several experimental studies have demonstrated their role in the mechanism of blood pressure regulation.

For example: Proceedings of the High Blood Pressure Research Council of Australia, Coronary Artery Baroreceptor-Mediated Changes In Arterial Pressure: A Pilot Study In Conscious And Anaesthetized Sheep, J S Bennetts, L F Arnolda, H C Cullen, J L Knight, R A Baker and D J McKitrick. The Bennetts study demonstrated that stretching of the proximal left anterior descending coronary artery with an inflatable balloon catheter elicited decreases in arterial pressure without changes in heart rate or electrocardiographic activity in anaesthetized sheep. Similar results were demonstrated in conscious sheep after surgical recovery of up to 2 weeks.

The referenced study supports the possibility that coronary artery baroreceptors exist and likely have a role in cardiovascular regulation.

CAB operate over much lower pressures and induce slower reflex vasoconstriction than the other baroreceptors and their resetting characteristics are also different. It is speculated in scientific literature that coronary baroreceptors play important role in the long term control of blood pressure. Although the coronary receptors are similar to other baroreceptors in that they exert a major control over vascular resistance, they do show several interesting differences. The most striking difference is their very low operating range; many are strongly active even at pressures as low as 60-80 mmHg. Other differences are that their reflex vascular response is not modified by changes in arterial pulsatility and that following a decrease in coronary pressure, the resulting vasoconstriction occurs at only about half the rate of the responses to decreases in carotid or aortic pressure. These differences in the behavior of the coronary baroreceptors raise the important issue of whether their function may also be different. One possibility on which several scientific publications have previously speculated is that they may be more concerned with the longer term control of blood pressure and this makes the subject of this invention potentially beneficial to the patients with sustained chronic hypertension.

Although role of CAB in blood pressure regulation is known it was never exploited for the benefit of patients with low or high blood pressure. There are several reasons for that. CAB are hard to access and located close to the heart. Electric stimulation in close proximity to the heart can cause dangerous arrhythmias. Mechanical distension of CAB with balloons was done previously but has little practical use since it interrupts vital blood flow to the heart muscle.

### SUMMARY

The authors noted that the phenomenon of the reduction of blood pressure in response to stretching CAB located in the walls of certain coronary arteries can be used to treat patients with hypertension. Authors propose the following novel methods and devices to make this physiologic phenomenon into a practical therapy:

A. A coronary artery is chosen that is known to contain baroreceptors and nerves conducting baroreceptor signals in its wall. Stimulation electrodes can be implanted a) outside of the coronary artery wall (extravascular), b) inside the coronary artery (intravascular) in contact with the wall or in a vein adjacent to the targeted artery (transvascular). Transvascular stimulation may have advantages over other modalities since venous access carries less risk of bleeding and thrombosis than arterial access and does not require invasive surgery.

B. Stimulation field can be applied to the targeted CAB using implanted electrodes during the refractory period of the heart when the heart tissue can not be easily excited and contract.

U.S. Pat. No. 6,522,926 to Kieval; Robert S. (Medina, Minn.) assigned to CVRx, Inc. (Maple Grove, Minn.) titled “Devices and methods for cardiovascular reflex control” describes in detail methods of intravascular and extravascular stimulation of carotid sinus baroreceptors that are applicable to coronary artery baroreceptors stimulation that is the subject of this invention. U.S. Pat. No. 6,522,926 is incorporated in this application in its entirety by reference.

It is understood that there are alternatives to stimulation CAB during the refractory period of the heart. So called sub-threshold stimulation is possible and known in the field of nerve stimulation. Sub-threshold stimulation takes advantage of the fact that the heart muscle tissue requires more energy to generate a contraction than some types of nerves. For example if the stimulation pulses are very short in the range of 50 to 150 microseconds they may be able to stimulate baroreceptors but not capture the heart muscle that typically requires pulses in the range of 0.25 to 1.0 ms to generate a contraction. This phenomenon can be used to stimulate CAB at any time in the cardiac cycle. It can be also envisioned that CAB can be stimulated with longer, more powerful pulses during the refractory period and at the reduced energy levels at other times. Authors chose stimulation during the refractory period for the disclosed embodiment since it allows application of higher energy and allows for an additional safety margin. For example: authors applied stimulation with very short duration of pulses of 50 microseconds at frequency of 200 Hz to the coronary blood vessels of an animal's heart. Such stimulation did not excite the heart and cause contractions at any time during the heart cycle but is known from scientific literature to excite nerve tissue and generate nerve signals. For the purpose of this disclosure electric stimulation applied to the heart is called non-excitatory electric stimulation if it does not cause contractions of the heart muscle.

Regardless of the modality CAB stimulation is expected to result in benefit to the patient primarily by peripheral vasodilatation and the reduction of blood pressure in hypertensive patients. CAB stimulation can be beneficial by reducing blood pressure in the group of patients with severe hypertension and particularly ones with malignant drug refractory hypertension that frequently results in strokes and sudden death.

Refractory Period Electric Stimulation

Inventors discovered that CAB stimulation is possible utilizing naturally occurring periods in the electric cyclical activity of the heart when the heart muscle conduction is blocked by so called refractory periods.

In the heart, there are specialized tissue collections that have a unique property; they rhythmically emit electrical impulses. The cause of these phenomena is the “leaky membrane” that allows the regular exchange primarily of Sodium and Potassium and to a lesser extent, Calcium and Chloride ions, that causes a change in the polarization of the cells. This sequence of depolarization and repolarization of tissue membranes is well known in the art. Briefly, sodium ions move into the cell and start the process of depolarization. At a certain point, potassium ions start move out of the cell and the repolarization of the cell begins. The most important aspect of the repolarization—depolarization cycle of the individual heart muscle elements that is relevant to this invention is the “refractory” period where the cells reset for the next wave and temporarily cannot be electrically stimulated to contract. Although the passive transmission of action potentials in these tissue elements would suggest that action potentials (e.g., the term used for the cycle of depolarization and repolarization in tissues) propagate in either direction along neighboring cells, most action potentials travel unidirectionally because the area behind the propagating action potential is unable to generate a repeat action potential for some period of time after the previous action potential has occurred.

This refractory period arises primarily because of the voltage-dependent inactivation of sodium channels, as described by Hodgkin and Huxley in 1952. In addition to the voltage-dependent opening of sodium channels, these channels are also inactivated in a voltage-dependent manner. Immediately after an action potential, during the absolute refractory period, virtually all sodium channels are inactivated and thus it is impossible to fire another action potential in that segment of membrane.

With time, sodium channels are reactivated in a stochastic manner. As they become available, it becomes possible to cause an action potential, albeit with a much higher threshold of energy required. This is the relative refractory period.

The sinoatrial node, atrial, atrioventricular node and ventricular tissues described in this patent all have absolute and relative refractory periods during which they are electrically and/or mechanically unable to respond to another electrical stimulus. Thus, a pacemaker impulse applied to these structures during these time points would either not cause or would required a significantly higher amount of energy delivered to cause electrically conduction and/or cause the heart muscle to contract.

The atria of the heart naturally contract during the approximately 100 ms following the P-wave of the surface ECG of the heart. The Q-wave of the surface ECG corresponds to the beginning of the absolute refractory period of the atria. The atria passively fill with blood during the ventricular systole, which occurs following the Q-wave of the surface ECG. Approximately half-way or 100-150 ms into the ventricular systole period, the atria are fully expanded. The ventricles contracts during the ventricular systole of the heart and are absolutely refractory to electric stimulation during this period. Atrial refractory period ends before the 50% of the ventricular systole has elapsed. The atria are now electrically and mechanically “armed” and can be triggered to contract by a pacing impulse. The end of the ventricular refractory period corresponds to the middle of the T-wave, which is also the time when the aortic valve opens.

In one embodiment of the invention, a sensing and pacing lead of a stimulator or pulse generator (that can be an implantable IPG or temporary) is placed in a great coronary vein (GCV) accessed through the coronary sinus (CS) in the position where the GCV is adjacent or substantially in proximity to the proximal left coronary artery of the heart. It is understood that the vascular anatomy of the human heart varies and in different cases different cardiac veins can be more suitable of the purpose of lead placement.

The target proximal left coronary artery can be, for example, the two major branches of the main left coronary artery Circumflex Artery (Cx) and Left Anterior Descending (LAD) Coronary Artery. Targeted CAB are likely to be present in the walls of the proximal LAD. Anatomically great cardiac vein often crosses these main brunches of the left coronary artery just distal of the location where the main left coronary artery branches into LAD and Cx. This junction is commonly referred to as bifurcation but can involve more than two branches.

The aorta is the major blood vessel that arises from the left ventricle and is separated from it by the aortic valve. The left main coronary artery arises from above the left portion of the aortic valve and then usually divides into two branches, known as the left anterior descending (LAD) and the circumflex (Cx) coronary arteries. In some patients, a third branch arises in between the LAD and the Circ. This is known as the ramus, intermediate, or optional diagonal coronary artery. The LAD travels in the groove (known as the inter-ventricular groove) that runs in the anterior or front portion the heart. It sits between the right and the left ventricles or the two lower chambers of the heart.

The LAD gives rise to the diagonal branches of the LAD that runs diagonally away from the LAD and towards the left edge in front of the heart.

The Circumflex (Cx) coronary artery is a branch of the left main coronary artery after the latter runs its course in between the aorta and the main pulmonary artery. The Cx travels in the left atrioventricular groove that separates the left atrium from the left ventricle. The Cx moves away from the Left Anterior Descending Artery (LAD) and wraps around to the back of the heart. The major branches that it gives off in the proximal or initial portion are known as obtuse marginal (OM) coronary arteries.

The heart ECG is sensed for signs of atrial and ventricular depolarization and repolarization. The beginning and end of the ventricular refractory period is predicted following a known delay after the P wave or R wave of the heart. For example, the CAB can be stimulated with a train of pulses approximately 50 milliseconds (ms) after the detected R wave. The desired delay can be recalculated by the embedded software based on the heart rate or set by the physician during the cathlab procedure or an office visit of the patient. All modern implantable pacemakers and IPGs include suitable sensing, programmability and telemetry functions.

It is understood that there are many ways to detect various phases of the electric heart activity cycle using surface or intracardiac ECG, pressures, wall motion or heart sound sensors. It is imagined that some of these signals can be used to synchronize the proposed stimulation to the desired window of the heart cycle. Common to all of these potential embodiments, the CAB are stimulated after the onset and before the end of the ventricular refractory period.

Electronic pacemakers are currently used to replace or supplement the natural pacing nodes of the heart by applying electric excitory signals to the heart muscle to cause contraction of one or more chambers of the heart and pump blood into the systemic and pulmonary circulations. Pacemakers are used in patients with a diseased sinoatrial node or other automatic tissues (slow heart beat) and defective (blocked) atrial or ventricular conduction pathways. Bi-ventricular pacemakers pace both ventricles of the heart to restore synchrony between the ventricles. It is understood that the methods and embodiments described in this patent may be incorporated into existing pacemaker devices, such as the pacemakers, biventricular pacemakers or ICDs.

### SUMMARY OF THE DRAWINGS

A preferred embodiment and best mode of the invention is illustrated in the attached drawings that are described as follows:

FIG. 1 illustrates an embodiment of the invention with a lead in coronary vein.

FIG. 2 illustrates the embodiment of the invention with an extravascular electrodes.

FIG. 3 illustrates timing of the refractory period pacing.

### DETAILED DESCRIPTION

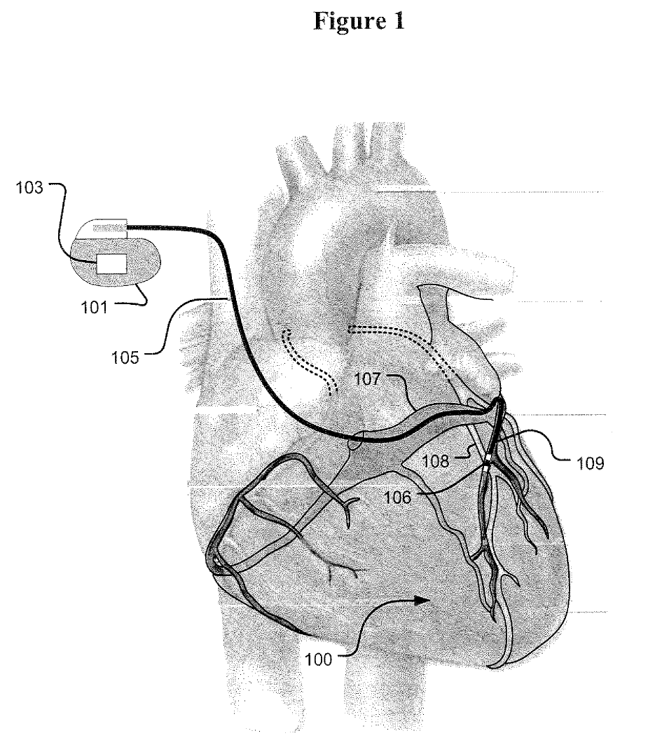

FIG. 1 shows a heart 100 treated with one embodiment of the invention. Implantable Pulse Generator (IPG) 101 is implanted in a tissue pocket in the patient's chest under the skin (not shown) using standard techniques similar to ones used to implant cardiac pacemakers. In this embodiment the IPG 101 is connected to the heart by one transvenous electrode lead 105. The lead 105 is equipped with one or more electrodes 106 that reside in a large coronary vein such as the coronary sinus 107. Methods of placing leads with electrodes in the coronary sinus and coronary veins are known and used in cardiac resynchronization therapy. Lead 105 can extend deeper into coronary veins and carry additional electrodes for adjunct therapies such as pacing of the left ventricle. Electrodes 106 can be used to apply stimulation signals and to sense electric signals of the heart ECG.

The placement of the electrodes 106 through the CS 107 is such that they are located in the Great Coronary Vein 109 close to the proximal LAD 108. It is believed that the proximal LAD 108 contains baroreceptors in its walls. It is believed that the unipolar or bipolar stimulation current applied by the IPG 101 to the electrodes 106 will create an electric field sufficiently strong to activate baroreceptors in the proximal LAD 108.

In the disclosed embodiment illustrated by the simplified diagram on the FIG. 1 electrodes 106 are implanted in the coronary sinus 107. It is understood that imaging studies and investigational stimulation with catheters and temporary leads can reveal different better suitable coronary veins such as the great cardiac vein 109 shown as an illustration of an alternative site for electrode placement.

An example of an implantable stimulator IPG is the Vagus Nerve Stimulation (VNS™) with the Cyberonics NeuroCybernetic Prosthesis (NCP®) System used for treatment of epilepsy. It is manufactured by Cyberonics Inc. IPGs from different manufacturers are virtually identical across application areas, usually varying only in the patterns of stimulating voltage pulses, style or number of electrodes used, and the programmed parameters. The basic implantable system consists of a pacemaker-like titanium case enclosing the power source and microcircuitry that are used to create and regulate the electrical impulses. An extension lead attached to this generator carries the electrical pulses to the electrode lead that is implanted or attached to the nerves or tissues to be stimulated.

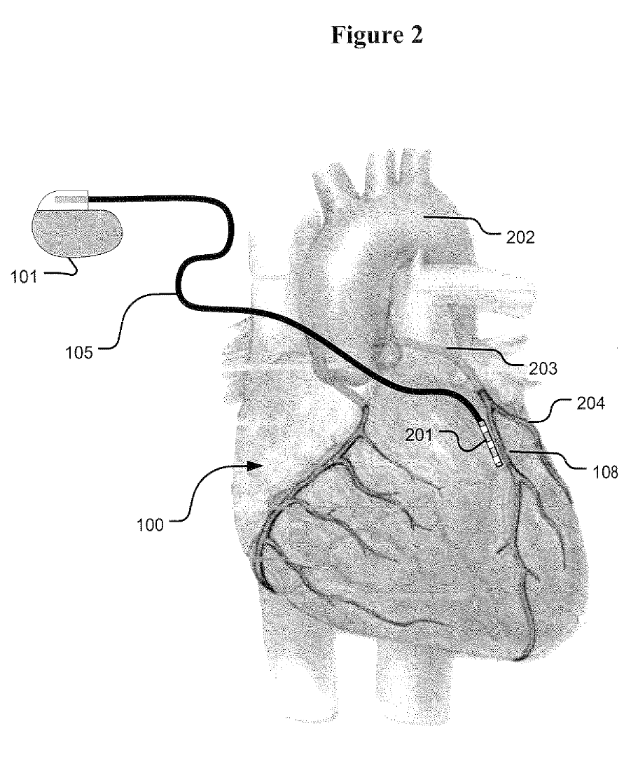

FIG. 2 illustrates an alternative embodiment of the invention. In this embodiment the stimulation electrodes 201 are placed extravascular and in close proximity to the proximal LAD 108. The IPG 101 and lead 105 can be similar to the transvascular embodiment but the lead is likely placed by a surgeon using sternotomy, thoracotomy or less invasive port access surgery. Skills needed to place electrodes near or around the LAD exist in the field of coronary bypass surgery.

The aorta 202 is the major blood vessel that arises from the left ventricle of the heart 100 and is separated from it by the aortic valve. The left main coronary artery 203 arises from above the left portion of the aortic valve and then divides into two branches, known as the left anterior descending (LAD) 108 and the circumflex (Cx) 204 coronary arteries.

During surgery the LAD 108 is exposed and an electrode lead can be attached to the surface of the heart by sutures. The electrode can have for example a shape of a paddle to overlap the artery and direct the stimulation energy towards baroreceptors. The paddle electrode can be made with an insulation backing and inserted between the surface of the heart and the LAD to reduce the electric energy delivered to the heart muscle and increase energy delivered to the baroreceptors in the wall of the LAD. Alternatively the electrode can be shaped as a flexible cuff and placed so that it is that it overlaps or wraps around the coronary artery. Clinically used spiral cuffs for connecting to a nerve are manufactured by Cyberonics Inc. (Houston, Tex.) that can be adopted for placement around an artery.

The wires, leads and the stimulator can be fully implanted at the time of surgery. Alternatively wires or leads can cross the skin and connect to the signal generator outside of the body. An implantable stimulator can be implanted later during a separate surgery or the use of an external stimulator can be continued.

The IPG can be also equipped with the sensor lead terminated with the sensor (not shown). The sensor can be a pressure sensor or an oxygen saturation sensor. The sensor can be located in the right ventricle of the heart, right atrium of the heart or other cavity of the heart. It can also be located outside of the heart in the aorta, the aortic arch or a carotid artery. If the sensor is a pressure sensor, it can be used to supply the IPG's intelligence with the information necessary to safely regulate the blood pressure. A venous blood oxygen saturation signal can be used in a similar way to control the stimulation based on oxygen demand. The sensor will be placed in the right atrium of the heart or in the vena cava. More than one sensor can be used in combination to supply information to the device. Sensors can be inside the vascular system (blood vessels) or outside of it. For example, a motion sensor can be used to detect activity of the person. Such sensor does not require placement outside the implanted device case and can be integrated inside the sealed case of the IPG as a part of the internal mechanism.

It is understood that the IPG can be also a cardiac pacemaker and can have more leads. It is expected that in future cardiac pacemakers will have even more leads connecting them to various parts of the anatomy. The leads can combine sensing and pacing electrodes as known and common in the field. The IPG 101 is equipped with the programmable logic that enables it to sense signals, process the information, execute algorithms and send out electric signals to the leads.

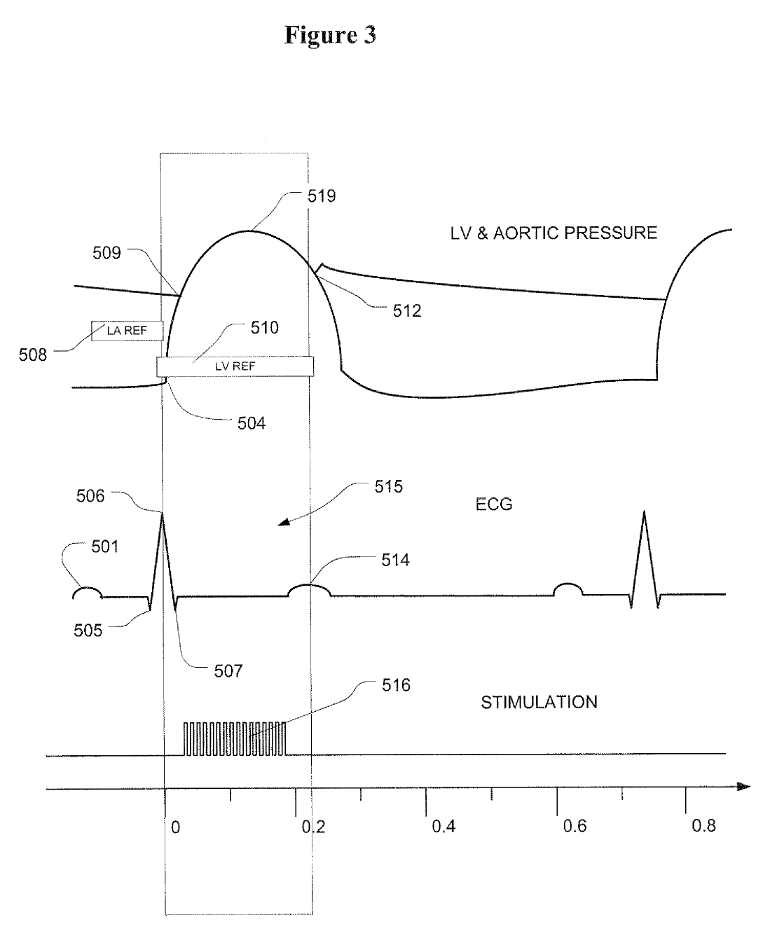

FIG. 3 illustrates stimulation of CAB with a sequence of stimulation pulses in relation to the timing of a heart cycle. Pulses are simplified and presented as a pulse burst 516 that consists of rectangular blocks spaced in time as represented by the X-axis.

The pulse burst can, for example, consist of individual unipolar and/or biphasic (of alternating polarity) pulses. Pulse duration can be chosen from values between 0.1 to 0.5 milliseconds and delivered at frequency of 5 to 100 Hz, based on the existing general experience with nerve stimulation, to elicit baroreflex. It is preferred to apply pulses of lowest possible amplitude and duration that will ensure the desired response without causing undesired activation of electrical or mechanical activity of the tissues. As previously noted, the amount of energy required to cause these undesired stimulations varies depending if the tissues are in the absolute or relative refractory period. It may be desired to alter the stimulation pattern during the pulse burst such that the energy delivered is higher during the absolute refractory period and is automatically reduced during the relative refractory period. The amount of energy delivered an be altered by changing either the pulse duration, pulse amplitude or both. The duration of the absolute and relative refractory periods can be automatically determined, such using a percentage of the period between heart beats, or user-set. Other methods, such as having a second lead in the ventricle to determine if the energy delivered is conducted to the ventricle, are well know in the art and may also be used. The energy required depends on the impedance of tissue between the electrode and the baroreceptors and on the energy losses in the interface. Based on the existing experience, pulses in the range of 0.25 to 5.0 V should be sufficient to transvenously stimulate baroreceptors in the proximal LAD. It is desired to maintain amplitude below the level that can cause irregular heart beats (arrhythmias), inadvertent heart muscle contraction, skeletal muscle twitching and pain. It is possible to include means to adjust these parameters after the implantation, using the stimulator's telemetry capability.

If the stimulation source is the constant current source, the stimulation intensity's range can be, for example, about 0.5 mA to 50 mA. Both monophasic and biphasic waveforms potentially can be used. The amplitude and frequency may vary burst to burst or pulse by pulse—within the same burst of pulses—for a single burst waveform. The burst duration can be in the range of 0.1 to 0.25 seconds, the ultimate limiting factor being the duration of the ventricular refractory period.

The IPG intelligence, e.g., a microprocessor 103 housed in the implant, may adjust the stimulation burst shape, pulse shape, frequency of pulses and amplitude of pulses to set or control the blood pressure. The system may also adjust the rate of rise and fall of the pulse amplitude within the burst to create ramps of variable shape. The microprocessor or monitors the heart, such as by sensing electric signals from the heart, e.g., ECG signals, pressure signals from a pressure sensor or oxygen saturation signals from an oxygen saturation sensor in the heart or vascular system. The microprocessor executes an algorithm that determines the burst shape, pulse shape, frequency of pulses and/or amplitude of pulses based on the sensor input signals.

FIG. 3 also illustrates the concept of the refractory period stimulation. The heart (See FIG. 1) has intact electric conduction including a substantially normal physiologic A-V node conduction delay. Alternatively, without substantive change to the invention, the heart can be paced artificially using the device also as a cardiac pacemaker. In both cases, the ventricle of the heart will have a predictable and detectable refractory period.

Stimulation in this embodiment is implemented by electric stimulation with extravascular, intravascular or transvascular electrodes (See FIGS. 1 and 2). Sensing of the cardiac electric activity can be performed with the same leads or additional atrial or ventricular leads known in the field of pacemakers.

The natural pacemaker or SA node of the heart initiates the Heart cycle with the P wave 501 of the ECG that corresponds to the beginning of atrial contraction. It is also the beginning of the heart systole. During atrial contraction, atrial pressure increases and atrial volume decreases. The end of this time period corresponds to the beginning of the atrial refractory period 508. During this period, the atria can not be paced to contract.

The P wave 501 of the ECG is followed by the Q wave 505 that signifies the beginning of the isovolumic contraction of the ventricle. Ventricular pressure 504 rise begins rapidly. In response the Tricuspid and Mitral valves of the heart close. Ventricular refractory period 510 begins. At the end of isovolumic contraction 509 The Pulmonary and Aortic valves open and the ejection of blood from the ventricle begins. Ventricular pressure reaches its peak in the middle of systole 519. The atrium is passively filled with blood as it relaxes. Approximately by the middle of systole both heart atria are filled with blood and their refractory period 508 is over. Atria are primed for a new contraction while the ventricle is ejecting blood. A-V valves are closed. At the same time the ventricle is still refractory and will not start another contraction in response to a natural or artificial pacing stimulus. Heart waves Q 505, R 506 and S 507 are commonly used markers of the beginning of the isovolumic contraction and the beginning of ventricular ejection (S wave). All modern pacemakers are equipped with means to read and analyze the ECG that are suitable for this embodiment of the invention.

Systole ends when the aortic valve closes 512. Isovolumic relaxation of the ventricle starts. This point also corresponds to the middle of the T wave 514 of the ECG. Importantly for the invention, the middle of T wave 514 corresponds to the end of the absolute refractory period 510 of the ventricle. At the end of the T-wave, the Tricuspid and Mitral valves open and the atrium volume starts to drop as the blood starts to flow from the atria into ventricles to prime them for the next ventricular contraction and ejection.

For this embodiment, the window of CAB stimulation opportunity 515 starts after the end of the atrial refractory period 508 approximately at the time of the R-wave 506. During this window, the ventricular muscle cannot be paced into contraction with a pacemaker pulse.

Stimulation 516 can occur at approximately the middle of systole or approximately 50 ms following the detected R wave 506 and/or 150 ms after P wave 501 is detected. Both P-wave and R-wave can be used by themselves or in combination to trigger pacing 516. In response to stimulation 516 burst coronary artery baroreceptors are activated, peripheral vasodilatation occurs and the patient's blood pressure is reduced. The stimulation burst 516 can be repeatedly applied over sequential or spaces out heartbeats for the duration of therapy.

Significantly, the window 515 and the burst 516 are inside the ventricular refractory period 510. Pacing atria outside of that time period is not desired since it can cause an arrhythmia and a premature ventricular beat.

It is possible that some patients will not need or will not be able to tolerate continuous CAB stimulation. In such patients period of normal baroreflex activity can be followed by the period of stimulation followed again by the rest period. Switching between stimulated and natural modes can be based on timing, patient's activity or physiologic feedbacks. For example, the pattern of therapy using electrical stimuli can be intermittent of varying duration of in intervals of 1-30 minute durations occurring, for example, every several hours or several times per day.

The invention has been described in connection with the best mode now known to the applicant inventors. The invention is not to be limited to the disclosed embodiment. Rather, the invention covers all of various modifications and equivalent arrangements included within the spirit and scope of the appended claims.

We claim:

1. A method to reduce blood pressure in a human patient comprising:
implanting at least one stimulation electrode proximal to a coronary artery of the patient,
implanting an implantable pulse generator in the patient, and
applying a non-excitatory electric stimulation to coronary artery baroreceptors.

2. The method of claim 1 wherein the non-excitatory stimulation stimulates the baroreceptors without cardiac muscle contraction or cardiac arrhythmia of the heart.

3. The method of claim 1 wherein the stimulation signal is applied during a refractory period of an electrical cycle the heart.

4. The method of claim 3 further comprising increasing energy of the stimulation signal during the refractory period.

5. The method of claim 4 further comprising reducing the energy of the stimulation signal during periods of the electrical cycle other than the refractory period.

6. The method of claim 1 wherein the stimulation signal is a sub-threshold stimulation signal.

7. A method to reduce blood pressure in a human patient comprising:
implanting at least one stimulation electrode proximal to a coronary artery of the patient, and
applying a non-excitatory electric stimulation signal using the stimulation electrode to stimulate a coronary artery baroreceptor in the coronary artery.

8. The method of claim 7 wherein the stimulation signal is applied during a refractory period of an electrical cycle the heart.

9. The method of claim 7 wherein the stimulation signal does not stimulate substantial cardiac muscle contraction or cardiac arrhythmia of the heart.

10. The method of claim 7 wherein the electrode is implanted adjacent an outside surface of a wall of the coronary artery.

11. The method of claim 7 wherein the electrode is implanted inside the coronary artery.

12. The method of claim 7 wherein the electrode is implanted in a vein adjacent to the coronary artery.

13. The method of claim 7 wherein the stimulation signal has an intensity in a range of 0.5 milliamps to 50 milliamps.

14. The method of claim 7 wherein the stimulation signal has a burst duration in a range of 0.1 second to 0.25 second.

15. The method of claim 7 wherein the stimulation signal includes stimulation pulses each having a duration in a range of 50 to 150 microseconds.

16. The method of claim 7 wherein a start of the refractory period is detected by sensing an R-wave of the electrical cycle of the heart.

17. The method of claim 16 wherein the stimulation signal is initiated at approximately 50 milliseconds following the R-wave.

18. The method of claim 7 wherein the stimulation signal includes a burst of stimulation pulses, each of said pulses having a duration in a range of 50 to 150 microseconds.

19. An apparatus to reduce blood pressure in a human patient comprising:
a stimulation electrode adapted to be implanted proximal to a coronary artery of the patient;
a pulse generator in communication with the stimulation electrode, wherein the pulse generator receives a signal indicative of a condition of an electrical cycle of the heart and generates a stimulation signal applied to the coronary artery to stimulate a coronary artery baroreceptor.

20. The apparatus of claim 19 wherein the pulse generator generates the stimulation signal during a refractory period of an electrical cycle of the heart and the signal indicative of a condition is indicative of an onset of the refractory period.

21. The apparatus of claim 20 wherein the pulse generator is an implantable pulse generator and the apparatus includes a wire lead between the generator and electrode to provide the communication.

22. The apparatus of claim 20 wherein the stimulation signal has an intensity in a range of 0.5 milliamps to 50 milliamps.

23. The apparatus of claim 20 wherein the stimulation signal has a burst duration in a range of 0.1 second to 0.25 second.

24. The apparatus of claim 20 wherein the stimulation signal includes stimulation pulses each having a duration in a range of 50 to 150 microseconds.

25. The apparatus of claim 21 wherein a start of the refractory period is detected by sensing an R-wave of the electrical cycle of the heart and the stimulation electrode senses the R-wave for the pulse generator.

26. The apparatus of claim 25 wherein the stimulation signal is initiated at approximately 50 milliseconds following the R-wave.

27. The apparatus of claim 20 wherein the stimulation signal includes a burst of stimulation pulses, each of said pulses having a duration in a range of 50 to 150 microseconds.
